(19) United States
(12) Patent Application Publication
(10) Pub. No.: US 2026/0089904 A1
(43) Pub. Date: Mar. 26, 2026

(54) CONDUCTIVE FILM AND ELECTROMAGNETIC WAVE SHIELDING MATERIAL USING SAME

(71) Applicant: PANASONIC INTELLECTUAL PROPERTY MANAGEMENT CO., LTD., Osaka (JP)

(72) Inventor: Tomohiro FUKAO, Winchester, MA (US)

(73) Assignee: PANASONIC INTELLECTUAL PROPERTY MANAGEMENT CO., LTD., Osaka (JP)

(21) Appl. No.: 19/403,654

(22) Filed: Nov. 28, 2025

Related U.S. Application Data
(63) Continuation of application No. PCT/JP2024/019452, filed on May 27, 2024.
(60) Provisional application No. 63/469,655, filed on May 30, 2023.

(51) Int. Cl. H05K 9/00; A61N 1/16; G06F 1/16

(52) U.S. Cl. CPC H05K 9/0084; A61N 1/16; G06F 1/163

(57) ABSTRACT

One aspect of the present invention relates to a conductive film including: a thermosetting resin layer; and a conductive layer on at least a part of at least one surface of the thermosetting resin layer, in which the thermosetting resin layer has an elongation at break at 20° C. of 50% or more, a tensile modulus at 20° C. of 1.0 MPa or more and 200 MPa or less, and a storage modulus at 250° C. of 0.1 MPa or more and 200 MPa or less, a film thickness of the conductive layer is 0.1 μm or more and 3.0 μm or less, the conductive layer contains a metal complex, and the conductive layer has a surface resistivity of 1×10⁻²Ω/□ or more and 10 Ω/□ or less.

### TECHNICAL FIELD

The present invention relates to a conductive film and an electromagnetic wave shielding material using the conductive film.

### BACKGROUND ART

In order to prevent malfunction of an electronic device due to an electromagnetic wave leaking from an electric circuit or the like, electromagnetic interference, leakage of information due to interception of communication radio waves, and the like, an electromagnetic wave shielding material has been conventionally used.

As the electromagnetic wave shielding material, a shielding material in which a conductive layer is laminated on a base material is generally used. For example, Patent Literature 1 discloses an electromagnetic wave absorbing sheet having flexibility, in which a resistance film, a dielectric layer, and an electromagnetic wave shielding layer are sequentially laminated, and the resistance film is formed of a conductive organic polymer.

On the other hand, recently, an electromagnetic wave shielding material that is wearable or capable of being used as a skin patch is required. In such applications, stretchability of the shielding material is required, but since the electromagnetic wave absorbing sheet described in Patent Literature 1 has flexibility but does not have stretchability, a gap is formed when the sheet is to be conformed to minute irregularities and three-dimensional curved surfaces, and an electromagnetic wave cannot be effectively blocked.

Patent Literature 2 discloses an electromagnetic wave shielding material having an electromagnetic wave shielding property, the electromagnetic wave shielding material including a base material having stretchability, and a conductive layer arranged on a surface of the base material. A thickness of the conductive layer is 5 nm or more and 500 nm or less, when tensile force is applied to the electromagnetic wave shielding material so that the electromagnetic wave shielding material extends by 2%, the conductive layer is cracked, and when tensile force is applied to the electromagnetic wave shielding material so that the electromagnetic wave shielding material extends by 2% and then the tensile force applied to the electromagnetic wave shielding material is released, a cracked portion of the conductive layer comes into contact again. In this technique, the conductive layer is formed on the stretchable base material, but when the sheet material is bent or in a case where the sheet material is to be conformed to sharp irregularities, the conductive layer is cracked and conductivity is deteriorated, and a sufficient blocking characteristic may not be obtained.

The present invention has been made in view of such actual circumstances, and an object of the present invention is to provide a conductive film which can be used as an electromagnetic wave shield and is excellent in conformability to irregularities and stretchability.

### CITATION LIST

### Patent Literature

Patent Literature 1: WO 2018/088492 A
Patent Literature 2: JP 6562834 B2

### SUMMARY OF INVENTION

As a result of intensive studies, the present inventor found that the problem can be solved by a laminate having the following configuration, and completed the present invention by conducting further studies based on this finding.

That is, a conductive film according to one aspect of the present invention is a conductive film including a thermosetting resin layer and a conductive layer on at least a part of at least one surface of the thermosetting resin layer, in which the thermosetting resin layer has an elongation at break at 20° C. of 50% or more, a tensile modulus at 20° C. of 1.0 MPa or more and 200 MPa or less, and a storage modulus at 250° C. of 0.1 MPa or more and 200 MPa or less, a film thickness of the conductive layer is 0.1 μm or more and 3.0 μm or less, the conductive layer contains a metal complex, and the conductive layer has a surface resistivity of 1×10⁻²Ω/□ or more and 10 or less.

### BRIEF DESCRIPTION OF DRAWINGS

FIG. 1 is a schematic cross-sectional view of a conductive film according to an embodiment of the present invention.

FIG. 2 is a schematic cross-sectional view of the conductive film according to another embodiment of the present invention.

FIG. 3 is a schematic cross-sectional view of the conductive film according to still another embodiment of the present invention.

FIG. 4 is a schematic cross-sectional view of the conductive film according to still another embodiment of the present invention.

FIG. 5 is a schematic cross-sectional view of the conductive film according to still another embodiment of the present invention.

### DESCRIPTION OF EMBODIMENTS

The conductive film of the present embodiment has a thermosetting resin layer and a conductive layer on at least a part of at least one surface of the thermosetting resin layer. The thermosetting resin layer has an elongation at break at 20° C. of 50% or more and a tensile modulus at 20° C. of 1.0 MPa or more and 200 MPa or less. Furthermore, the thermosetting resin layer has a storage modulus at 250° C. of 0.1 MPa or more and 200 MPa or less. A film thickness of the conductive layer is 0.1 μm or more and 3.0 μm or less, and the conductive layer contains a metal complex. Further, the conductive layer has a surface resistivity of 1× 10⁻²Ω/□ or more and 10Ω/□ or less.

In the conductive film of the present embodiment, as described above, since the thermosetting resin layer has an elongation at break at 20° C. of 50% or more, the thermosetting resin layer has an elongation can efficiently block an electromagnetic wave without losing conductivity even in an irregular portion having a small radius of curvature or the like. Further, since the thermosetting resin layer has a tensile modulus at 20° C. of 1.0 MPa or more and 200 MPa or less, the thermosetting resin layer has flexibility and is capable of conforming to irregularities and curved surfaces without a gap. Then, since a storage modulus of the thermosetting resin layer at 250° C. is 0.1 MPa or more and 200 MPa or less, the film has sufficient heat resistance. Further, since the conductive layer contains a metal complex and has a film thickness of 0.1 μm or more and 3.0 μm or less, breakage of the conductive layer due to elongation of the film can be suppressed while sufficient conductivity and an electromagnetic wave blocking property are provided.

Therefore, the conductive film of the present embodiment is a conductive film that can be suitably used as an electromagnetic wave shield and is excellent in conformability to irregularities and stretchability.

Hereinafter, a specific embodiment of the present invention will be described with reference to the drawings and the like. Note that the embodiment described below is only one of various embodiments of the present invention. The embodiment below can be modified in various ways depending on design as long as the object of the present invention can be achieved. Further, reference signs in the drawings denote the following: 1 conductive film, 2 conductive layer, 3 resin layer, 4 adhesive layer.

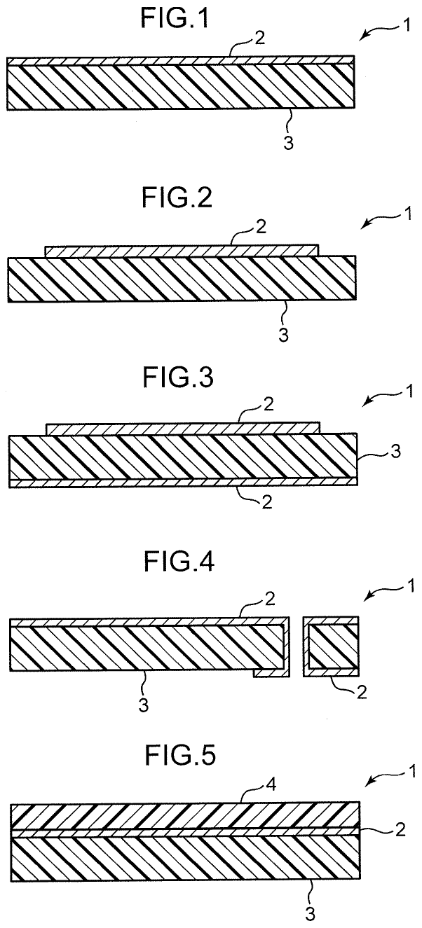

As illustrated in FIG. 1, a conductive film 1 of the present embodiment includes a thermosetting resin layer 3 and a conductive layer 2 on at least a part of at least one surface of the thermosetting resin layer 3. The conductive layer 2 may be provided on the entire surface of one surface of the thermosetting resin layer 3 as illustrated in FIG. 1, or may be provided only on a part of one surface of the thermosetting resin layer 3 as illustrated in FIG. 2. Alternatively, as illustrated in FIG. 3, the conductive layer 2 can be provided on both surfaces of the thermosetting resin layer 3. In the conductive film 1 illustrated in FIG. 3, the conductive layer 2 is provided on the entire surface of one surface and a part of another surface of the thermosetting resin layer 3, but the present invention is not limited to such an embodiment, and the conductive layer 2 may be provided on the entire surfaces of both surfaces or a part of both surfaces.

In another embodiment, the conductive film 1 of the present embodiment can also include the conductive layer 2 inside the thermosetting resin layer 3 as illustrated in FIG. 4. In the specific example illustrated in FIG. 4, the conductive layer 2 is provided on a surface of a through hole provided in the thermosetting resin layer 3. With such a configuration, there is also an advantage that the conductive layers provided on both surfaces can be electrically connected, and connected to an electrical ground with a small area.

Each configuration of the conductive film according to the present embodiment will be described more specifically.

### (Thermosetting Resin Layer)

The thermosetting resin layer (hereinafter, also simply referred to as “resin layer”) of the present embodiment has stretchability, has an elongation at break at 20° C. of 50% or more, and a tensile modulus at 20° C. of 1.0 MPa or more and 200 MPa or less.

In the present embodiment, the elongation at break refers to the elongation until breakage, and is an index indicating flexibility of the resin layer together with the tensile modulus. A more preferable elongation at break is 100% or more and 500% or less. An upper limit of the elongation at break in the present embodiment is preferably as high as possible, but 1000% is sufficient.

The thermosetting resin layer has a tensile modulus at 20° C. of 1.0 MPa or more and 200 MPa or less, and the tensile modulus is more preferably 1.0 MPa or more and 50 MPa or less and still more preferably 1.5 MPa or more and 30 MPa or less.

With the conductive film including the resin layer having an elongation at break and a tensile modulus in the above-described ranges, conformability at the time of deformation into an arbitrary shape is high, and therefore, for example, it is possible to obtain a conductive film and an electromagnetic shielding material which have excellent conformability to clothing and the like, are hardly broken, and are excellent in stretchability.

A tensile modulus of the present embodiment is a value measured by the following method:

First, a cured product of a resin composition constituting the resin layer is cut into a size of 50 mm×5.5 mm and attached to a universal testing machine (AGS-X manufactured by Shimadzu Corporation). Then, the test is conducted at room temperature (20° C.) and a tension speed of 500 mm/min, and the slope of r-σ (initial tensile modulus) is determined from all the stress (σ) data corresponding to the strain (r) at 1.0% to 5.0% elongation by the least squares method to calculate the tensile modulus.

$$Strain ( r ) = x / x 0 ( x is the gripper moving distance , x 0 is the initial distance between grippers )$$

$$Stress ( σ ) = F / ( d \cdot l ) ( F is test force , d is film thickness , and l is width of a test piece )$$

Further, regarding the elongation at break, the elongation when cured product is fractured is measured using the testing machine.

Further, the thermosetting resin layer of the present embodiment has a storage modulus at 250° C. of 0.1 MPa or more and 200 MPa or less. By the above, the resin layer of the present embodiment can ensure sufficient heat resistance, and for example, can withstand temperature of a drying step or a curing step of ink or the like when a conductive layer is formed using the ink. A more preferable range of the storage modulus is 1 MPa or more and 10 MPa or less. The storage modulus in the present embodiment is a value that can be measured by the method described in an example described later.

The resin layer of the present embodiment is not particularly limited as long as the resin layer is composed of a material (thermosetting resin composition) containing thermosetting resin in which a tensile modulus, an elongation at break, and a storage modulus are within the above ranges, but for example, the resin layer is preferably formed of any of a cured product, a semi-cured product, or a dried product of a resin composition containing thermosetting resin. Note that, in the present embodiment, the “semi-cured product” is one in a state in which the resin composition is partially cured and may be further cured. That is, the semi-cured product is a resin composition in a semi-cured state (B-staged). For example, when a resin composition is heated, viscosity of the resin composition first gradually decreases, then curing starts, and the viscosity gradually increases. In such a case, semi-curing includes a state between after the viscosity starts to increase and before the resin composition is completely cured, and the like. Further, in the present embodiment, the “dried product” means a resin composition before curing (the resin composition in A stage) in which a solvent is dried.

As the thermosetting resin, for example, thermosetting resin generally used as a base material of various films or an insulating layer of an electronic base material can be used. Specific examples of the thermosetting resin include epoxy resin, polyrotaxane resin, acrylic resin, urethane resin, silicone resin, isocyanate resin, polyol resin, and hydrogenated styrene based elastomer resin. Among them, the resin layer of the present embodiment is preferably any of a cured product, a semi-cured product, or a dried product of a resin composition containing at least one thermosetting resin selected from epoxy resin, polyrotaxane resin, acrylic resin, and hydrogenated styrene based elastomer resin.

The thermosetting resin is particularly preferably epoxy resin. Specific examples of the epoxy resins include bisphenol A type epoxy resin, bisphenol F type epoxy resin, bisphenol S type epoxy resin, aralkyl epoxy resin, phenol novolac type epoxy resin, alkylphenol novolac type epoxy resin, biphenol type epoxy resin, naphthalene type epoxy resin, dicyclopentadiene type epoxy resin, an epoxidized product of a condensate of a phenol and an aromatic aldehyde having a phenolic hydroxyl group, triglycidyl isocyanurate, and alicyclic type epoxy resin. One type of these may be used alone or two or more types of these may be used in combination depending on a situation.

As the epoxy resin, more preferably, for example, epoxy resin containing two or more epoxy groups in one molecule and having a molecular weight of 500 or more, epoxy resin having an epoxy equivalent of 400 g/eq or more, or the like is exemplified. As such epoxy resin, a commercially available one may be used, and examples of it include JER1003 (manufactured by Mitsubishi Chemical Corporation, molecular weight 1300, bifunctional), EXA-4816 (manufactured by DIC, molecular weight 824, bifunctional), YP50 (manufactured by NIPPON STEEL Chemical & Material Co., Ltd., molecular weight 60,000 to 80,000, bifunctional), PMS-14-67 (manufactured by Nagase ChemteX Corporation, molecular weight 300,000, multifunctional), and PASR-001 (manufactured by Nagase ChemteX Corporation, molecular weight 500,000, multifunctional). Further, one type of the epoxy resin as described above may be used alone, or two or more types of the epoxy resin may be used concurrently.

Polyrotaxane resin that can be suitably used in the present embodiment is resin having a structure in which a linear axial molecule passes through a cyclic molecule and ends are blocked to prevent the cyclic molecule from coming off. Specifically, polyrotaxane such as one described in U.S. Pat. No. 4,482,633 is mentioned.

One type of the thermosetting resin as described above can be used alone or two or more types of the thermosetting resin can be used in combination.

In the thermosetting resin composition used in the resin layer of the present embodiment, a mixed amount of the thermosetting resin as described above is not particularly limited as long as the thermosetting resin serves as a main agent, but is usually preferably about 30% by mass or more and 98% by mass or less with respect to the entire amount of the resin composition. A more preferable mixed amount is 50% by mass or more and 90% by mass or less.

The thermosetting resin composition used in the present embodiment may further contain a curing agent for the purpose of curing the thermosetting resin. Examples of the curing agent include phenolic curing agents, amine based curing agents, acid anhydride based curing agents, ester based curing agents, imidazole based curing agents, dicyandiamide, cationic curing agents, and metal soaps. These can be used without particular limitation, and one type or more of these may be used concurrently.

In the present embodiment, the mixed amount of the curing agent is not particularly limited as long as the curing agent serves as a curing agent, but is preferably about 0.1% by mass or more and 30% by mass or less with respect to the entire amount of the resin composition. A more preferable mixed amount is 0.1% by mass or more and 10% by mass or less.

Furthermore, the resin composition of the present embodiment may contain various additives as other components other than the above. Examples of the additive include a curing accelerator, a dispersant, a filler, an antioxidant, a leveling agent, a pigment, and a dye agent. One or a plurality of types of these may be used concurrently. The mixed amount of these other components can be appropriately set depending on their roles, and is not particularly limited, but is preferably about 0.001 parts by mass or more and 50% by mass or less with respect to the entire amount of the resin composition.

A thickness of the resin layer of the present embodiment is not particularly limited as long as the resin layer serves as a base material (base layer) of the conductive film, but the thickness is preferably about 10 μm or more and 500 μm or less from the viewpoint of having both properties of flexibility and strength as a support body when used for an electromagnetic wave shielding material. The thickness is more preferably 25 μm or more and 200 μm or less, still more preferably 50 μm or more and 100 μm or less.

A method of forming the resin layer of the present embodiment is not particularly limited, but as an example, first, a thermosetting resin composition constituting the resin layer is dissolved in an appropriate organic solvent (for example, methyl ethyl ketone, toluene, and the like) to prepare resin varnish. Then, the resin varnish is applied using a bar coater or the like onto a support body such as a PET film so as to have a desired thickness, and dried to remove the solvent, so that the resin layer can be formed. Furthermore, the resin layer may be formed by performing heating and pressurization under a predetermined condition to cure the resin composition.

### (Conductive Layer)

In the conductive film of the present embodiment, the conductive layer contains a metal complex, and the conductive layer has a surface resistivity of 1×10⁻²Ω/□ or more and 10Ω/□ or less. By providing such a conductive layer with a film thickness of 0.1 μm or more and 3.0 μm or less, the conductive film of the present embodiment can efficiently block an electromagnetic wave. A more preferable range of the surface resistivity is 1×10⁻²Ω/□ or more and 1×10⁻¹Ω/□ or less.

In the present embodiment, the metal complex contained in the conductive layer is a compound formed by bonding of a metal ion to one or more ligands. In the present embodiment, the conductive layer can be formed by, for example, performing a reduction reaction, a substitution reaction, an oxidation-reduction reaction, thermal decomposition, or the like using a metal complex. The reason why the conductive layer obtained from a metal complex is rich in stretchability is not clear, but the conductive layer does not have a regular crystal structure and has an amorphous or fine crystal structure. On the other hand, when compared with a conductive layer formed by another method of obtaining a metal thin film, for example, a vacuum vapor deposition method or a sputtering method, the conductive layer of the present embodiment is slightly inferior in conductivity, but has sufficient conductivity for a specific application, for example, shielding of an electromagnetic wave. Furthermore, since the conductive layer is rich in stretchability, the conductive film of the present embodiment has sufficient conformability to irregularities and stretchability, and is excellent in workability for various products (including a flexible cloth product). In addition, since the conductive layer of the present embodiment contains a metal complex, there is an excellent advantage that, even when the conductive film is repeatedly stretched, a crack (fracture) and the like are less likely to occur as in a conventional conductive layer, and conductivity can be maintained even after stretching.

In the present embodiment, the metal complex is obtained, for example, by reacting a metal salt with a catalyst. Examples of the method of producing a metal complex include a method in which a metal salt and a catalyst are added to a dissolving agent and stirred for predetermined time. The stirring method is not particularly limited, and can be appropriately selected from publicly-known methods, for example, a stirring method using a stirrer, a stirring blade, or a mixer, a method of adding an ultrasonic wave, and the like.

In the present embodiment, examples of the metal constituting a metal complex include iron, cobalt, nickel, copper, zinc, ruthenium, rhodium, palladium, silver, platinum, osmium, iridium, and the like. Among them, silver is preferably contained from the viewpoint of more excellent conductivity.

In the present embodiment, examples of the metal salt for forming a metal complex include a metal oxide, a chloride, a sulfide, a cyanide, a carbonate, an acetate, a nitrate, a subacid salt, a sulfate, a phosphate, and a carboxylate. Among them, a carboxylate is preferable from the viewpoint of more excellent conductivity and storage stability.

In the present embodiment, a metal complex can be obtained by reducing a silver salt to silver metal with a catalyst. Examples of the catalyst include a metal borohydride salt, an aluminum hydride salt, an amine compound, an alcohol, an organic acid, a reducing sugar, a sugar alcohol, sodium sulfite, a hydrazine compound, dextrin, hydroquinone, hydroxylamine, ethylene glycol, glutathione, and an oxime compound. Among them, the amine compound is preferable from the viewpoint of more excellent reaction rate and stability. Examples of the amine compound include a primary amine, a secondary amine, a tertiary amine, a polyamine, and the like, and for example, methylamine, ethylamine, propylamine, butylamine, pentylamine, hexylamine, heptylamine, octylamine, nonylamine, decylamine, undecylamine, dodecylamine, tridecylamine, tetradecylamine, pentadecylamine, hexadecylamine, heptadecylamine, octadecylamine, allylamine, n-propylamine, isopropylamine, n-butylamine, sec-butylamine, tert-butylamine, n-pentylamine, isopentylamine, 2-ethylhexylamine, tert-hexylamine, phenylamine, cyclopentylamine, tert-octylamine, tert-decylamine, tert-dodecylamine, tert-octadecylamine, dimethylamine, diethylamine, dipropylamine, dibutylamine, diphenylamine, dicyclopentylamine, methylbutylamine, trimethylamine, triethylamine, tripropylamine, triphenylamine, ethylenediamine, 1,3-diaminopropane, hexamethylenediamine, and the like are included.

In the present embodiment, the metal complex may contain a dissolving agent. The dissolving agent is selected from a group including an organic solvent, a chelating agent, and a combination of these. The organic solvent is selected from a group including alkane hydrocarbon, carbamate, alkene, cyclic hydrocarbon, aromatic hydrocarbon, amine, polyamine, amide, ether, ester, alcohol, thiol, thioether, phosphine, and a combination of these. Examples of the organic solvent include pentane, hexane, heptane, octane, nonane, decane, undecane, dodecane, tridecane, tetradecane, pentadecane, hexadecane, octadecane, nonadecane, eicosane, cyclohexane, cycloheptane, cyclooctane, cyclononane, cyclodecane, decalin, benzene, toluene, xylene, tetralin, xylene, dimethyl ether, diethyl ether, dipropyl ether, dibutyl ether, methyl t-butyl ether, tetrahydrofuran, tetrahydropyran, dihydropyran, 1,4-dioxane, 1-propanol, 2-propanol, 1-methoxy-2-propanol, 1-butanol, 2-butanol, 1-pentanol, 2-pentanol, 3-pentanol, 1-hexanol, 2-hexanol, 3-hexanol, 1-octanol, 2-octanol, 3-octanol, tetrahydrofurfuryl alcohol, cyclopentanol, terpineol, terpineol, and the like. Examples of the chelating agent include ethylenediaminetetraacetic acid, iminodiacetic acid, ethylenediamine-di(o-hydroxyphenylacetic acid), nitrilotriacetic acid, dihydroxyethylglycine, trans-1,2-cyclohexanediaminetetraacetic acid, diethylenetriamine-N,N,N′,N″,N″-pentaacetic acid, glycol ether diamine-N,N,N′,N′-tetraacetic acid, dimethyl sulfoxide, diethylenetriamine, tert-octylamine, tert-butylamine, 2-ethylhexylamine, ethylenediamine, and the like.

The conductive layer of the present embodiment can be formed by a metal complex ink containing the metal complex as described above. The metal complex ink is preferably an ink containing the metal complex in a content of 0.1 wt % or more and 40 wt % or less.

The metal complex ink that can be used in the present embodiment may be a commercially available product, and for example, silver complex ink “EI-1207” manufactured by Electroninks Inc. and the like are suitably used.

The conductive layer of the present embodiment only needs to contain the metal complex at least partially. Such a conductive layer can be obtained, for example, by applying the metal complex ink as described above onto the thermosetting resin layer by a spraying method or the like so that the film thickness after formation is 0.1 μm or more and 3.0 μm or less, and heating and sintering (performing sintering) at a predetermined temperature (depending on a type of metal).

### (Adhesive Layer)

The conductive film 1 of the present embodiment may further include an adhesive layer 4 as illustrated in FIG. 5. The adhesive layer 4 can serve as an adhesive layer when the conductive film 1 is bonded to an object and also serve as a protective layer of the conductive layer 2.

The adhesive layer 4 may be provided on the conductive layer 2 as illustrated in FIG. 5, but may also be provided on the thermosetting resin layer 3. Further, the adhesive layer 4 may be provided on both the conductive layer 2 and the thermosetting resin layer 3. By providing the adhesive layer 4, there is an advantage that the conductive film can be easily adhered to an adherend. In particular, providing the adhesive layer 4 on the conductive layer 2 is preferable because there is an advantage that the conductive layer can be effectively protected.

The adhesive layer 4 in the present embodiment is not particularly limited as long as the adhesive layer 4 is formed of a material having adhesive property.

Specifically, as the adhesive layer 4 of the present embodiment, an adhesive, a pressure sensitive adhesive, or the like used in the field of an electromagnetic wave shielding material or the like can be used without particular limitation.

Examples of the adhesive that can be used in the present embodiment include a thermoplastic adhesive, a thermosetting adhesive, and the like. More specifically, the thermoplastic adhesive is, for example, an adhesive containing polyvinyl alcohol, acrylic resin, polyvinyl acetate, polyethylene, polyolefin, polyamide, polyester, various rubbers, or polyurethane as a main component, or the like. As the thermosetting adhesive, for example, an epoxy resin based adhesive, a phenol resin based adhesive, or the like can be used. The thermosetting adhesive may be composed of a semi-cured product of resin or the like.

Examples of the pressure sensitive adhesive include a rubber based pressure sensitive adhesive, an acryl based pressure sensitive adhesive, a silicone based pressure sensitive adhesive, and a urethane based pressure sensitive adhesive. From the viewpoint of adhesion, an acryl based pressure sensitive adhesive or a urethane based pressure sensitive adhesive is preferable.

The thickness of the adhesive layer in the present embodiment is not particularly limited, but is preferably 0.1 μm or more and 200 μm or less, still more preferably 0.5 μm or more and 50 μm or less from the viewpoint of achieving both properties of bonding strength and flexibility.

A method of forming the adhesive layer 4 is not particularly limited, and for example, in a case where an adhesive or the like is used, the adhesive layer 4 can be formed by applying the adhesive to a desired thickness on the conductive layer 2 and/or the thermosetting resin layer, or by bonding a film shaped adhesive film, pressure sensitive adhesive film, or the like.

### (Application of Conductive Film)

The conductive film as described above can be used in various applications, and particularly can be very suitably used as an electromagnetic wave shielding material.

### (Electromagnetic Wave Shielding Material)

The present embodiment also includes an electromagnetic wave shielding material composed of the above-described conductive film. The electromagnetic wave shielding material of the present embodiment can be arranged on minute irregularities or a three-dimensional curved surface without a gap, and can effectively block an electromagnetic wave. Further, there is an advantage that the electromagnetic wave shielding material can be attached to any shape and is excellent in workability.

Since the electromagnetic wave shielding material of the present embodiment has the above characteristic, the electromagnetic wave shielding material is suitably used for a wearable computing device. Specifically, for example, the electromagnetic wave shielding material can be suitably used in a scene or an application where a bioelectric signal is detected, such as a myoelectric sensor, an electrocardiogram sensor, an electroencephalogram sensor, or the like.

The present description discloses techniques in various aspects as described above, and a main technique among them is summarized below.

A conductive film according to a first aspect of the present invention is a conductive film including:
a thermosetting resin layer; and
a conductive layer on at least a part of at least one surface of the thermosetting resin layer, in which
the thermosetting resin layer has an elongation at break at 20° C. of 50% or more, a tensile modulus at 20° C. of 1.0 MPa or more and 200 MPa or less, and a storage modulus at 250° C. of 0.1 MPa or more and 200 MPa or less,
a film thickness of the conductive layer is 0.1 μm or more and 3.0 μm or less,
the conductive layer contains a metal complex, and
the conductive layer has a surface resistivity of 1×10⁻²Ω/□ or more and 10Ω/□ or less.

A conductive film according to a second aspect of the present invention is the conductive film of the first aspect, in which the conductive layer is formed using a metal complex ink.

A conductive film according to a third aspect of the present invention is the conductive film of the first or second aspect, in which the metal complex contains silver.

A conductive film according to a fourth aspect of the present invention is the conductive film of any of the first to third aspects, further including a conductive layer also inside the thermosetting resin layer.

A conductive film according to a fifth aspect of the present invention is the conductive film of any of the first to fourth aspects, further including an adhesive layer.

A conductive film according to a sixth aspect of the present invention is the conductive film according to any of the first to fifth aspects, in which the thermosetting resin layer is any of a cured product, a semi-cured product, and a dried product of a resin composition containing at least one thermosetting resin selected from an epoxy resin, a polyrotaxane resin, an acrylic resin, and a hydrogenated styrene based elastomer resin.

An electromagnetic wave shielding material according to a seventh aspect of the present invention is an electromagnetic wave shielding material consists of the conductive film according to any of the first to sixth aspects.

An electromagnetic wave shielding material according to an eighth aspect of the present invention is the electromagnetic wave shielding material according to the seventh aspect that is used in a wearable computing device.

Hereinafter, the present invention will be described more specifically with reference to Examples, but the scope of the present invention is not limited to these.

### EXAMPLES

First, various materials used in the present example are as follows.

### (Epoxy Resin)

### Epoxy Resin 1

Acrylonitrile as the polymerization unit (a), isobornyl acrylate as the polymerization unit (b), and a polymerization unit (c) represented by the following formula (1) were polymerized such that the blending ratio (polymerization %) of (a):(b):(c) was 10:20:70, and further, glycidyl methacrylate as the polymerization unit (d) was added such that the epoxy equivalent with respect to the total amount of the acrylic resin was the numeral value given in Table 1. After that, the mixture was subjected to a polymerization reaction, affording epoxy resin 1 containing methyl ethyl ketone as a solvent (“PMS-14-67EK40” manufactured by Nagase ChemteX Corporation). The solid ratio was 40% by weight.

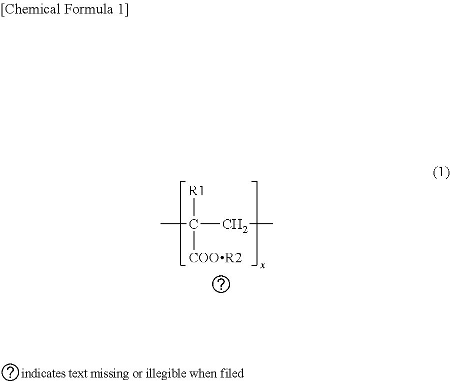

(where, R1 is hydrogen or a methyl group and R2 is hydrogen or an alkyl group. X represents an integer.)

### Epoxy Resin 2

Epoxy resin 2 (“PMS-14-64EK40” manufactured by Nagase ChemteX Corporation) was obtained in the same manner except that the amount of the polymerization unit (d) of the “PMS-14-67EK40” was changed. The solid ratio was 40% by weight.

### Epoxy Resin 3

Epoxy resin 3 (“PASR-001” manufactured by Nagase ChemteX Corporation) was obtained in the same manner except that the ratio of the polymerization units (a) to (d) of the “PMS-14-67EK40” was changed. The solid ratio was 20%.
Bisphenol type epoxy resin (“jER1003” manufactured by Mitsubishi Chemical Corporation)
Bisphenol type epoxy resin (“jER1006F S” manufactured by Mitsubishi Chemical Corporation)

### (Polyrotaxane)

Polyrotaxane; “SH3400P” manufactured by ASM Inc.

### (Curing Agent)

Acid anhydride curing agent (“RIKACID TBN-100” of New Japan Chemical Co., Ltd.)
Amine based curing agent (“POREA SL-100A” manufactured by Kumiai Chemical Industry Co., Ltd.)
Carboxylic acid based curing agent (“TN-1” manufactured by NOF Corporation)·
Phenolic curing agent (“KAYAHARD GPH-103” manufactured by Nippon Kayaku Co., Ltd.)
Acid anhydride curing agent (“YH-307” manufactured by Mitsubishi Chemical Corporation)

### (Curing Accelerator)

Imidazole based curing accelerator (“2PZ-CN” manufactured by Shikoku Chemicals Corporation)

### (Solvent)

Methyl ethyl ketone

### (Metal Ink)

Silver complex ink; “EI-1207” manufactured by Electroninks Inc.
Palladium particle containing ink; “METALLOID ML-130” manufactured by lox Co., Ltd.

### <Preparation of Thermosetting Resin Layer (Cured Film)>

Resin varnishes 1 to 9 were prepared using each component in a blending composition (parts by mass) shown in Table 1. After standing defoaming, the resin varnishes 1 to 9 were applied to a PET film (SP-PET O1 manufactured by Mitsui Chemicals Tohcello, Inc.) using a bar coater so as to have a thickness (after curing) shown in Table 2 described later. Subsequently, the film was heated in an oven at 80° C. for 60 minutes and further heated at 180° C. for 120 minutes to obtain cured resin films 1 to 11 to be a thermosetting resin layer. Note that a thickness of each cured resin film was measured with a micrometer (MDH-25 MB manufactured by Mitutoyo Corporation).

TABLE 1

| Resin varnish | | Varnish 1 | Varnish 2 | Varnish 3 | Varnish 4 | Varnish 5 | Varnish 6 | Varnish 7 | Varnish 8 | Varnish 9 |
|---|---|---|---|---|---|---|---|---|---|---|
| Epoxy resin | PMS-14-67EK40 | 78.88 | 84.60 | 86.63 | 87.90 | | | | 100.00 | |
| | PMS-14-64EK40 | | | | | 90.19 | | | | |
| | PASR-001 | | | | | | 90.23 | | | |
| | jER1003 | | | | | | | 18.4 | | |
| | jER1006FS | | | | | | | | | 33.81 |
| Polyrotaxane | SH3400P | | | | | | | 24.9 | | |
| Curing agent | TBN-100 | 8.05 | | | | 3.53 | 3.52 | | | 15.69 |
| | SL-100A | | 5.77 | | | | | | | |
| | TN-1 | | | 8.26 | | | | | | |
| | GPH-103 | | | | 4.45 | | | | | |
| | YH-307 | | | | | | | 6.5 | | |
| Curing accelerator | 2PZ-CN | 0.40 | 0.40 | 0.40 | 0.40 | 0.40 | 0.40 | 0.40 | | 0.50 |
| Solvent | Methyl ethyl ketone | 12.67 | 9.23 | 4.71 | 7.25 | 5.88 | | 49.85 | | 50.00 |

### <Characteristic of Thermosetting Resin Layer (Cured Film)>

### (Methods of Measuring Tensile Modulus and Elongation at Break)

A sample (cured resin film) was cut into a size of 90 mm*5.5 mm and attached to a universal tester (AGS-X manufactured by Shimadzu Corporation), a test was performed at a tensile speed of 500 mm/min, and a tensile modulus was calculated from a stress at an elongation from 1.0% to 5.0%. Further, an elongation at the time when the sample was broken was measured. These tensile modulus and elongation at break were measured at room temperature (20° C.).

### (Method of Measuring Storage Modulus)

Each cured resin film was cut into a 10 mm×30 mm piece and attached to a dynamic viscoelasticity analyzer (DMS6100 manufactured by Seiko Instruments Inc.). A test was conducted at a strain amplitude of 10 μm, a frequency of 10 Hz (sine wave), and a rate of temperature increase of 5° C./min, and a storage modulus at 250° C. was measured.

A result is presented in Table 2. Note that since a cured film 10 was melted by heating, the storage modulus could not be measured.

TABLE 2

| | Cured | Cured | Cured | Cured | Cured | Cured | Cured | Cured | Cured | Cured | Cured |
|---|---|---|---|---|---|---|---|---|---|---|---|
| | film 1 | film 2 | film 3 | film 4 | film 5 | film 6 | film 7 | film 8 | film 9 | film 10 | film 11 |
| Varnish | Varnish 1 | Varnish 2 | Varnish 3 | Varnish 4 | Varnish 5 | Varnish 6 | Varnish 7 | Varnish 7 | Varnish 7 | Varnish 8 | Varnish 9 |
| Film thickness | 103 | 104 | 106 | 100 | 103 | 99 | 101 | 25 | 204 | 105 | 98 |
| [μm] | | | | | | | | | | | |
| Tensile modulus | 14.3 | 6.6 | 17.4 | 29.8 | 3.0 | 132.1 | 4.1 | 4.4 | 4.0 | 0.8 | 203.5 |
| [MPa] | | | | | | | | | | | |
| Elongation at | 144.9 | 176.9 | 140.4 | 133.6 | 364.9 | 108.1 | 218.4 | 222.5 | 208.8 | 739.9 | 41.2 |
| break [%] | | | | | | | | | | | |
| 250° C. storage | 1.4 | 1.1 | 1.4 | 1.2 | 0.1 | 57 | 0.6 | 0.7 | 0.6 | Unmea- | 230 |
| modulus [MPa] | | | | | | | | | | surable | |

### <Production of Conductive Film>

### Examples 1 to 11 and Comparative Examples 1 to 4

Each cured film to be a thermosetting resin layer was placed on a platen heated to 200° C., and a silver complex ink was applied by a spray method so as to have a uniform film thickness with the thickness shown in Table 3 so as to form a conductive layer to obtain a conductive film of Examples 1 to 11 and Comparative Examples 1 to 4.

### Comparative Example 5

A palladium particle containing ink was applied onto the cured film 7 with a bar coater so as to have a uniform film thickness of 0.7 μm, and then heated in an oven at 80° C. for 30 minutes to dry the ink. Further, a conductive layer was formed by electroless copper plating to obtain a conductive film of Comparative Example 5.

### Comparative Example 6

The cured film 7 was introduced into a vacuum deposition apparatus, and copper was deposited so as to have a thickness of 0.1 μm to form a conductive layer, and a conductive film of Comparative Example 6 was obtained.

### Comparative Example 7

The cured film 7 was introduced into a sputtering apparatus, and sputtering was performed so that titanium had a thickness of 0.005 μm and then copper had a thickness of 0.1 μm to form a conductive layer, and a conductive film of Comparative Example 7 was obtained.

### (Thickness of Conductive Layer)

A film thickness of the conductive layer in the conductive film was measured by cutting the conductive film of each of Examples and Comparative Examples into a 3 mm square, polishing a cross section of the cut conductive film fixed with embedded resin with an ion milling apparatus, and observing the cross section of the conductive film with an optical microscope.

### [Evaluation Test]

### <Method of Measuring Sheet Resistance>

For each conductive film, a value of a film thickness was input to a resistivity meter (MCP-T370, manufactured by Mitsubishi Chemical Analytech Co., Ltd.), and a volume resistivity of a surface of the conductive film was measured by a four terminal method. An average value of five or more measurement results was employed as a volume resistivity.

### <Method of Evaluating Heat Resistance>

A storage modulus at 250° C. of the conductive film was measured in the same manner as the method of measuring a storage modulus, and evaluated according to the following criteria.
Pass 1 MPa or more
Fail Less than 1 MPa or less than measurement limit

### <Method of Evaluating Electromagnetic Wave Shielding Property>

Each conductive film was installed in an electromagnetic wave shielding property evaluation apparatus described in Japanese Patent No. 5619265, and an electric field shielding effect was measured. Then, an electric field shielding effect from 1 GHz to 6 GHz was evaluated according to the following criteria.

| | |
|---|---|
| Excellent | 30 dB or more |
| Good | 20 dB or more and less than 30 dB |
| Fair | 10 dB or more and less than 20 dB |
| Fail | Less than 10 dB |

### <Method of Evaluating 10% Repeated Stretchability>

Each conductive film was cut to a size of 10 mm×40 mm, and installed on a stretchability evaluation apparatus (DMLHB, manufactured by YUASA SYSTEM Co., Ltd.) and a planar body tensile test jig (DLD-ST, manufactured by YUASA SYSTEM Co., Ltd.) so that a fixed distance was 20 mm. A wiring connected to a resistance meter (RM3545-02, manufactured by HIOKI E.E. CORPORATION) was fixed to both ends of a test piece with an alligator clip. The test piece was repeatedly expanded and contracted at a cycle of 0.19 Hz while a resistance value at 10% expansion and contraction is measured, and the number of times of expansion and contraction until the resistance value reached 1200Ω was measured. Evaluation was performed according to the following criteria based on the number of times of expansion and contraction.

| | |
|---|---|
| Excellent | 10,000 times or more |
| Good | 5000 times or more and less than 10,000 times |
| Fair | 1000 times or more and less than 5000 times |
| Fail | Less than 1000 times |

The above results are summarized in Tables 3 and 4.

TABLE 3

| | Exam- | Exam- | Exam- | Exam- | Exam- | Exam- | Exam- | Exam- | Exam- | Exam- | Exam- |
|---|---|---|---|---|---|---|---|---|---|---|---|
| | ple 1 | ple 2 | ple 3 | ple 4 | ple 5 | ple 6 | ple 7 | ple 8 | ple 9 | ple 10 | ple 11 |
| Film | Cured | Cured | Cured | Cured | Cured | Cured | Cured | Cured | Cured | Cured | Cured |
| | film 1 | film 2 | film 3 | film 4 | film 5 | film 6 | film 7 | film 8 | film 9 | film 7 | film 7 |
| Conductive layer | Silver | Silver | Silver | Silver | Silver | Silver | Silver | Silver | Silver | Silver | Silver |
| | complex | complex | complex | complex | complex | complex | complex | complex | complex | complex | complex |
| | ink | ink | ink | ink | ink | ink | ink | ink | ink | ink | ink |
| Conductive layer | Spray | Spray | Spray | Spray | Spray | Spray | Spray | Spray | Spray | Spray | Spray |
| forming method | | | | | | | | | | | |
| Conductive layer | 0.7 | 0.7 | 0.7 | 0.7 | 0.7 | 0.7 | 0.7 | 0.7 | 0.7 | 0.1 | 3.0 |
| film thickness | | | | | | | | | | | |
| [μm] | | | | | | | | | | | |
| Sheet resistance | 0.8 | 0.8 | 0.8 | 0.8 | 0.8 | 0.8 | 0.8 | 0.8 | 0.8 | 9 | 0.03 |
| [Ω/sq] | | | | | | | | | | | |
| Heat resistance | Excel- | Excel- | Excel- | Excel- | Excel- | Excel- | Excel- | Excel- | Excel- | Excel- | Excel- |
| | lent | lent | lent | lent | lent | lent | lent | lent | lent | lent | lent |
| Electromagnetic | Excel- | Excel- | Excel- | Excel- | Excel- | Excel- | Excel- | Excel- | Excel- | Good | Excel- |
| wave shielding | lent | lent | lent | lent | lent | lent | lent | lent | lent | | lent |
| property | | | | | | | | | | | |
| 10% repeated | Excel- | Excel- | Excel- | Excel- | Excel- | Excel- | Excel- | Excel- | Good | Good | Good |
| stretchability | lent | lent | lent | lent | lent | lent | lent | lent | | | |

TABLE 4

| | Comparative | Comparative | Comparative | Comparative | Comparative | Comparative | Comparative |
|---|---|---|---|---|---|---|---|
| | Example 1 | Example 2 | Example 3 | Example 4 | Example 5 | Example 6 | Example 7 |
| Film | Cured film 10 | Cured film 11 | Cured film 7 | Cured film 7 | Cured film 7 | Cured film 7 | Cured film 7 |
| Conductive layer | Silver | Silver | Silver | Silver | Copper/ | Copper | Copper/ |
| | complex ink | complex ink | complex ink | complex ink | palladium | | titanium |
| Conductive layer | Spray | Spray | Spray | Spray | Electroless | Vacuum | Sputtering |
| forming method | | | | | plating | deposition | |
| Conductive layer film | 0.7 | 0.7 | 0.05 | 4.0 | 0.7 | 0.1 | 0.1 |
| thickness [μm] | | | | | | | |
| Sheet resistance [Ω/□] | 0.8 | 0.8 | 54 | 0.01 | 0.1 | 0.3 | 0.07 |
| Heat resistance | Fail | Excellent | Excellent | Excellent | Excellent | Excellent | Excellent |
| Electromagnetic wave | Excellent | Excellent | Fail | Excellent | Excellent | Excellent | Excellent |
| shielding property | | | | | | | |
| 10% repeated | Excellent | Fail | Fair | Fail | Fail | Fail | Fail |
| stretchability | | | | | | | |

### (Discussion)

As clear from the result in Table 3, it was confirmed that all of the conductive films of the present embodiment had low resistivity, excellent heat resistance, and excellent electromagnetic wave shielding property. Further, it was also found that increase in resistivity can be suppressed and conductivity can be maintained even when 10% expansion and contraction are repeated. That is, it was shown that the conductive film of the present embodiment is a conductive film which can be used as an electromagnetic wave shield and is excellent in conformability to irregularities and stretchability.

On the other hand, as shown in Table 4, in the conductive film of Comparative Example 1 in which the thermosetting resin layer did not have a sufficient storage modulus, heat resistance was not obtained, and in the conductive film of Comparative Example 2 in which the thermosetting resin layer did not have a sufficient elongation at break, conductivity after expansion and contraction could not be maintained. Further, in the conductive film of Comparative Example 3 in which thickness of the conductive layer was too thin, sufficient electromagnetic wave shielding property could not be obtained, and conductivity after expansion and contraction was also poor as a result. In the conductive film of Comparative Example 4 in which thickness of the conductive layer was too large, conductivity after expansion and contraction could not be maintained. Further, in the conductive films of Comparative Examples 5 to 7 in which a conductive layer was formed by a method (plating, vapor deposition, sputtering) used for forming a conventional conductive layer without using a metal complex, a crack was generated in the conductive layer when the conductive film was stretched, and conductivity after expansion and contraction could not be maintained.

In order to express the present invention, the present invention has been described above appropriately and sufficiently through the embodiments with reference to specific examples, drawings and the like. However, it should be recognized by those skilled in the art that changes and/or improvements of the above-described embodiments can be readily made. Accordingly, changes or improvements made by those skilled in the art shall be construed as being included in the scope of the claims unless otherwise the changes or improvements are at the level which departs from the scope of the appended claims.

### INDUSTRIAL APPLICABILITY

The present invention has wide industrial applicability in technical fields related to electromagnetic wave absorption, electromagnetic wave shielding, and the like.

1. A conductive film comprising:
a thermosetting resin layer; and
a conductive layer on at least a part of at least one surface of the thermosetting resin layer, wherein
the thermosetting resin layer has an elongation at break at 20° C. of 50% or more, a tensile modulus at 20° C. of 1.0 MPa or more and 200 MPa or less, and a storage modulus at 250° C. of 0.1 MPa or more and 200 MPa or less,
a film thickness of the conductive layer is 0.1 μm or more and 3.0 μm or less,
the conductive layer contains a metal complex, and
the conductive layer has a surface resistivity of 1×10⁻²Ω/□ or more and 10Ω/□ or less.

2. The conductive film according to claim 1, wherein the conductive layer is formed using metal complex ink.

3. The conductive film according to claim 1, wherein the metal complex contains silver.

4. The conductive film according to claim 1, further comprising a conductive layer also inside the thermosetting resin layer.

5. The conductive film according to claim 1, further comprising an adhesive layer.

6. The conductive film according to claim 1, wherein the thermosetting resin layer is any of a cured product, a semi-cured product, and a dried product of a resin composition containing at least one thermosetting resin selected from an epoxy resin, a polyrotaxane resin, an acrylic resin, and a hydrogenated styrene based elastomer resin.

7. An electromagnetic wave shielding material consists of the conductive film according to claim 1.

8. The electromagnetic wave shielding material according to claim 7 that is used in a wearable computing device.
